(19) United States
(12) Patent Application Publication
(10) Pub. No.: US 2017/0105653 A1
(43) Pub. Date: Apr. 20, 2017

(54) BIO-IMPEDANCE MEASUREMENT METHOD USING BI-PHASIC CURRENT STIMULUS EXCITATION FOR IMPLANTABLE STIMULATOR

(71) Applicant: THE REGENTS OF THE UNIVERSITY OF CALIFORNIA, Oakland, CA (US)

(72) Inventors: Wentai Liu, Los Angeles, CA (US); Yi-Kai Lo, Los Angeles, CA (US); Chih-Wei Chang, Los Angeles, CA (US)

(73) Assignee: THE REGENTS OF THE UNIVERSITY OF CALIFORNIA, Oakland, CA (US)

(21) Appl. No.: 15/336,652

(22) Filed: Oct. 27, 2016

Related U.S. Application Data
(63) Continuation of application No. PCT/US2015/028063, filed on Apr. 28, 2015.
(60) Provisional application No. 61/985,583, filed on Apr. 29, 2014.

(51) Int. Cl. A61B 5/053; A61N 1/36; A61B 5/00; A61B 5/0215; A61B 5/11

(52) U.S. Cl. CPC A61B 5/0538; A61B 5/02158; A61B 5/1107; A61B 5/4238; A61B 5/4255; A61B 5/6873; A61B 5/6876; A61N 1/36007; A61B 5/4836; A61N 1/36135

(57) ABSTRACT

Method and apparatus for estimating bio-impedance at electrode-electrolyte interface by injecting a single low-intensity bi-phasic current stimulus having an selected inter-pulse delay first and second current pulse phases, which involves acquiring transient electrode voltage along the bi-phasic current stimulus waveform. Determining equivalent circuit parameters of an electrode, at the electrode-electrolyte/tissue interface, based on transient electrode voltage across said multiple temporal locations is also performed.

### CROSS-REFERENCE TO RELATED APPLICATIONS

This application is a 35 U.S.C. §111(a) continuation of PCT international application number PCT/US2015/028063 filed on Apr. 28, 2015, incorporated herein by reference in its entirety, which claims priority to, and the benefit of, U.S. provisional patent application Ser. No. 61/985,583 filed on Apr. 29, 2014, incorporated herein by reference in its entirety. Priority is claimed to each of the foregoing applications.

The above-referenced PCT international application was published as PCT International Publication No. WO 2015/168162 on Nov. 5, 2015, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technological Field

This technical disclosure pertains generally to electrical stimulators, and more particularly to determining bio-impedance for an electrical stimulator.

2. Background Discussion

The proper application of functional electrical stimulators relies on having some knowledge of the bio-impedance at the electrode-electrolyte/tissue interface. Impedance can also be utilized as a merit to: (1) evaluate the proximity between electrodes and targeted tissues, (2) estimate the safe boundary of the stimulation parameters, and/or (3) be used as a biomarker to monitor the activity of internal organs (i.e., contraction/relaxation of smooth muscles in intestine/colon/stomach) or tension of blood vessels.

One simple approach for estimating bio-impedance is based on the injection of a small sinusoidal current at a fixed frequency and the measurement of the evoked voltage at the electrode. However, this approach can only provide the information of the impedance at a given frequency without having an equivalent circuit model available.

In another approach, electrochemical impedance spectroscopy (EIS) has been widely used to derive electrode-electrolyte impedance. EIS is based on the pseudo-linearity characteristic of the electrode and a small AC potential (typically between 1 and 10 mV) is applied to excite the electrochemical cell. Nonetheless, the electrode-electrolyte/tissue impedance is not linear. Thus, doubling of excitation voltage might not necessarily double the applied current as expected, while stimulation usually evokes a large transient voltage at the electrode. Thus, EIS does not appear to be the best approach for impedance measurement of stimulation electrodes. In addition, the hardware cost of the EIS approach is high, with additional complexity being required when integrating EIS into a neural stimulator.

Bio-impedance measurement based on voltage/current pulse excitation has been proposed to infer the parameters of a three-element Randles cell electrode model. One of these proposals involves injecting a current stimulus into the electrode and measuring the resulting voltage, but only the electrode-tissue resistance can be derived. A sophisticated computation is presented in one approach, the complexity of which impeded it from being incorporating into implantable stimulators. One of these methods is capable of acquiring all parameters of a Randles cell, but a prerequisite is to deliver a stimulus with infinite pulse width to the electrode; which is both problematic to achieve and would cause an electrode overpotential higher than its water window. Therefore it is seen that numerous attempts have been made with little success in regards to determining bio-impedance.

Accordingly, a need exists for a workable solution for determining bio-impedance at the electrode-electrolyte/tissue interface.

### BRIEF SUMMARY

Obtaining information about equivalent circuit parameters of an electrode is useful in a number of regards, such as electrode placement and stimulus signal generation. By utilizing equivalent circuit parameters, a safe boundary can be set for stimulus parameters in order not to exceed the water window of electrodes. An impedance measuring technique is presented with an implemented proof-of-concept system using an implantable neural stimulator and an off-the-shelf processing element (e.g., microcontroller). The technology presented yields the parameters of an electrode equivalent circuit by injecting a single low-intensity bi-phasic current stimulus, in the range of several microamps (μA) to tens of microamps, with deliberately inserted inter-pulse delay and by acquiring the transient electrode voltage at three well-specified timing intervals.

Use of a low-intensity stimulus allows the derivation of electrode double layer capacitance since capacitive charge-injection dominates when electrode overpotential is small. Insertion of the interpulse delay creates a controlled discharge time to estimate the Faradic resistance. The method presented has been validated by measuring the impedance of (a) an emulated Randles cell made of discrete circuit components and (b) utilizing a custom-made platinum electrode array to compare estimated parameters with the results derived from an impedance analyzer.

The method presented herein can be integrated into implantable or commercial neural stimulator systems with a low overhead in regards to power consumption, hardware cost, and computation. Current commercial neural stimulators can only measure electrode impedance at a given frequency. By contrast, the present disclosure yields circuit parameters which aid in determining proximity between electrodes and tissue, but also for setting stimulus parameters to prevent electrode damage.

In the present disclosure, excitation is based on using a bi-phasic current pulse with interpulse delay. The technique utilizes the electrode characteristic themselves, in which pure capacitive charge-injection dominates the initial electric charge transfer from the electrode to the tissue when the electrode overpotential is small and the faradic charge transfer process does not happen. A deliberately specified period of interpulse delay is then applied to acquire parameters of a Randles cell model of an electrode with simple computation and low hardware cost. The range of the inserted interpulse delay is mainly dependent on the size of the electrode that determines its discharge time constant, and the resolution of the off-the-shelf processing unit (i.e., microprocessor). The length of the interpulse delay must be set to ensure the decayed electrode overpotential is larger than the minimal resolution of the quantizer (i.e., analog-to-digital converter). As a rule of thumb, the maximal interpulse delay can be set as approximately 2.8 times the electrode discharge time constant.

In one embodiment, the presented technology adopts a bi-phasic current stimulus excitation to yield the parameters of the equivalent circuit model of an electrode without complex computation and hardware setup. In addition, the presented technology can be conveniently integrated into commercial systems with little extra hardware overhead, since modern stimulators are typically designed to allow for the use of generating bi-phasic current stimulus in driving an electrode. The presented technology is applicable to a wide range of stimulators, and is also applicable to implantable stimulators for prosthetic devices.

In one embodiment, to monitor the propagating activity of internal organs along the gastrointestinal track (i.e., stomach, intestine, colon) or tension of the vascular smooth muscles, simultaneous multi-site stimulation on multiple electrodes placed on top of the tissue can be performed to measure the bio-impedance change in real-time. It is important to note that stimulus delivered to these electrodes must be time-interleaved to ensure the delivered current does flow to the ground/reference electrode, instead of flowing into adjunct stimulation electrodes. The above setup enables the measurement of propagating slow waves of the gastrointestinal track or blood pressure for a closed-loop implantable stimulator. It can also be used in clinical studies on the enteric/autonomic nervous system.

Further aspects of the presented technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The disclosed technology will be more fully understood by reference to the following drawings which are for illustrative purposes only:

FIG. 1 is a diagram of electrode placement in the human body, such as may utilize an embodiment of the present disclosure.

FIG. 2 are plots of impedance for a plurality of electrodes as seen in FIG. 1, as utilized within an embodiment of the present disclosure.

FIG. 3A through FIG. 3C are a schematic and waveforms diagrams associated with a Randles cell, step current stimulus, and electrode voltage waveforms.

FIG. 4A and FIG. 4B are waveform diagrams of a bi-phasic current stimulus within interpulse delay (FIG. 4B), and induced voltage at the electrode (FIG. 4A) which is utilized for determining parameters of a Randles cell according to at least one embodiment of the present disclosure.

FIG. 5A and FIG. 5B are a schematic of a multi-channel neural stimulator utilizing a system-on-chip (SoC), which determines bio-impedance according to at least one embodiment of the present disclosure.

FIG. 6A and FIG. 6B are waveform diagrams of electrode response to bi-phasic current stimulus within interpulse delay at two different intensity levels, according to at least one embodiment of the present disclosure.

FIG. 7A through FIG. 7C are images of a 3×9 platinum polyimide electrode array utilized for testing a bio-impedance measurement according to at least one embodiment of the present disclosure.

FIG. 8A and FIG. 8B are plots of estimated circuit parameters of an electrode comparing varied pulse width and intensity, determined according to at least one embodiment of the present disclosure.

FIG. 9 is a flow diagram of a method for determining bio-impedance according to at least one embodiment of the present disclosure.

### DETAILED DESCRIPTION

### 1. Introduction

Significant benefit is derived from electrode-stimulus applications when the impedance of the electrode-electrolyte interface is understood. If the circuit parameters are known, the limit of stimulus intensity and pulse width can be determined in order not to exceed the water window of the electrode underuse and the compliance voltage of the stimulator. Characterizing the electrode-electrolyte interface by the present disclosure provides benefits for additional applications as well.

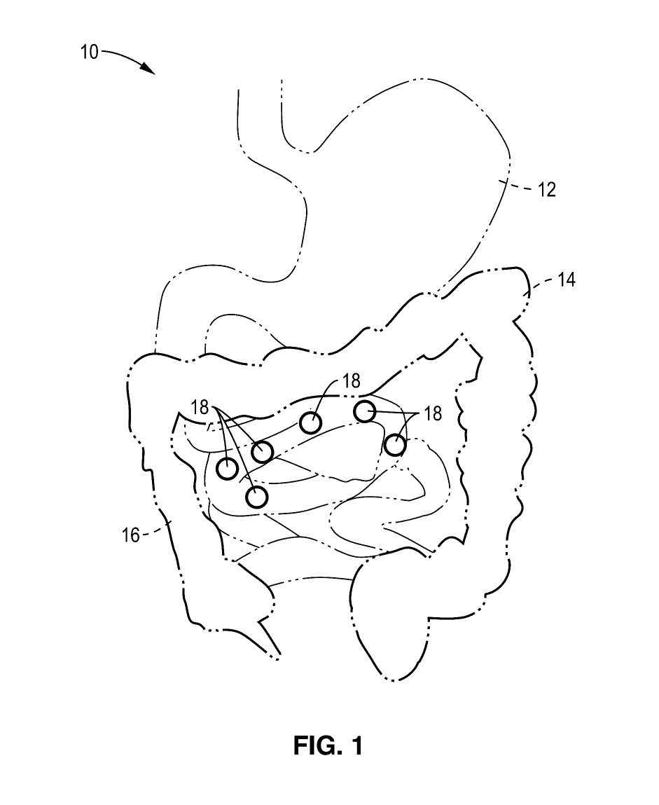

FIG. 1 illustrates an example embodiment 10 of applying the bio-impedance characterization method disclosed herein to electrodes 18 shown positioned at possible locations within the internal organs for tracking smooth muscle activity.

By applying these techniques to the normal or pathological smooth muscle of internal organs (e.g., stomach 12, large intestine 14, and small intestine 16), the contraction/relaxation of the muscle activities can be monitored through impedance change in the electrode-electrolyte interface.

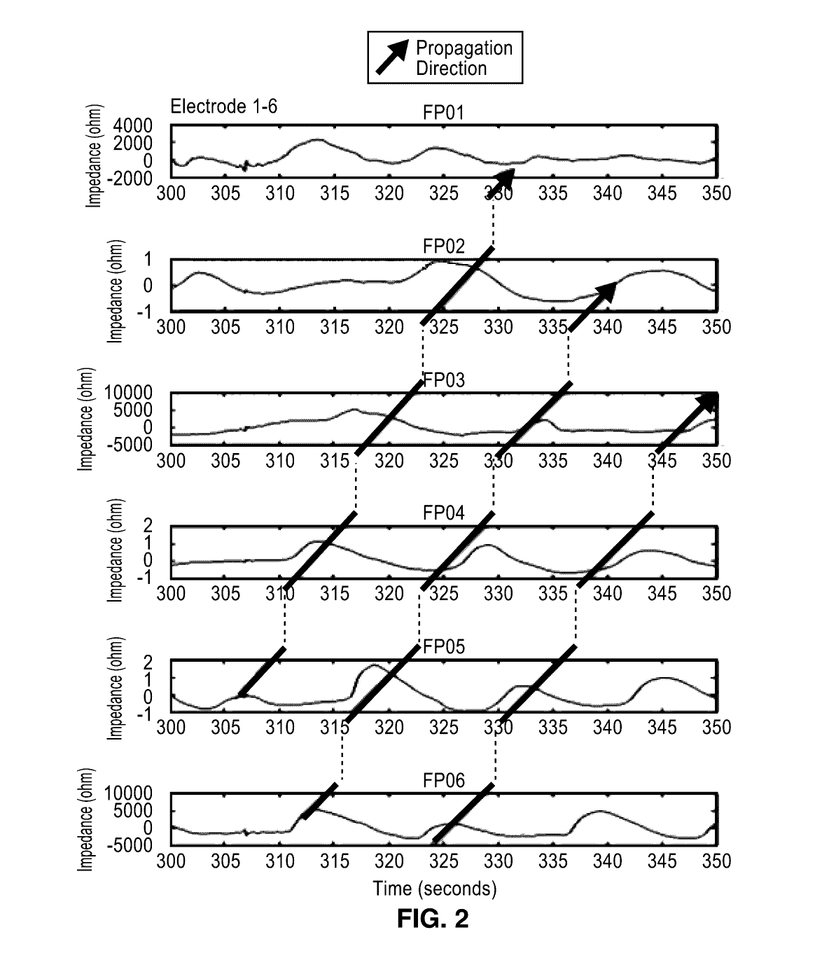

FIG. 2 depicts impedance changes representing the propagation of the slow-wave activity resulting from the smooth muscle contraction/relaxation for each of the six electrodes seen in FIG. 1A, with the propagation direction between channels shown by the arrows. It should be noted that electrode placement is not limited to locations depicted in FIG. 1A. By applying multiple-electrodes on the normal or pathological smooth muscle of internal organ (such as small and large intestine and stomach) and performing the disclosed bio-impedance measurements, the propagation of smooth muscle contraction/relaxation wave can be monitored in vivo and in vitro. This ability is of significant advantage as it is currently not feasible to monitor or record intestinal activity in vivo without damaging its smooth muscles or neural networks. In at least one embodiment, the measured impedance signal can be utilized as a feedback signal to one or more implantable devices for controlling drug delivery, or any desired means of stimuli (i.e., electrical, optical, magnetic, stimulation, and so on). In another embodiment, the same methodology can also be adopted to measure the pressure of the blood vessel, which can also be reflected from the bio-impedance variation of activating vascular smooth muscles. This serves as an alternative tool for recording smooth muscle activities, and can be performed non-invasively, in contrast to conventional methods that require inserting a pressure catheter into the target organ in order to measure a single point of pressure and thus multi-site activity monitoring with those systems is infeasible and unrealistic. The presented bio-impedance technology adopts small current, short stimulation pulses to ensure that the stimulation does not activate smooth muscle activity, while acquiring information on bio-impedance changes relating to smooth muscle activities. Moreover, the proposed method also enables the simultaneous electrical recording and stimulation through the same electrode. As stimulus with large pulse width and high intensity is usually used to activate neuron/muscles, the low-intensity and short stimulus used for bio-impedance measurement can be co-registered to the same electrode simultaneously while the artifact caused by strong stimulus can be filtered in frequency domain with ease.

Bio-impedance measurements according to the presented technology provides a number of important features. (a) Simple bi-phasic current excitation is utilized to measure bio-impedance, whereby the method is applicable for use in commercial neural stimulators. (b) Measurement based on bi-phasic current stimulus ensures the charge balance at the electrode, overcoming the problems with accumulated charge causing a DC offset at the electrode impacting the measurement of Faradic resistance when utilizing mono-phasic stimulation. (c) By leveraging the initial pure capacitive charging of the stimulating electrode, double layer capacitance can be readily estimated. (d) An interpulse pulse delay specified in the stimulus parameters enables the estimation of Faradic resistance. (e) The presented technique provides a way for users to set the stimulation parameters based on the electrode parameters estimated to avoid electrode or tissue damage. The following sections describe the details of this bio-impedance measurement method.

### 2. Voltage Transient on Electrodes

Electrical charge is delivered from an electrode through two main mechanisms: capacitive charge-injection and faradic charge injection. Bio-impedance can be schematically represented by an equivalent electrical circuit.

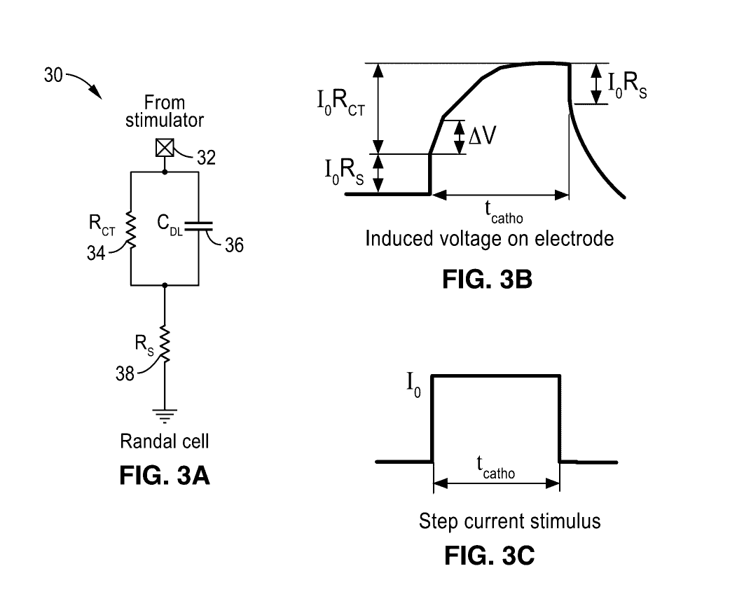

FIG. 3A illustrates an example embodiment 30 of a simple three-element Randles cell electrode-electrolyte model showing connection from stimulator 32 to a circuit consisting of a charge transfer resistance R_{CT} 34, a double layer capacitance C_{dl} 36, and a tissue-solution resistance R_{S} 38 shown connected to ground, is herein adopted since both mechanisms are incorporated.

FIG. 3B and FIG. 3C depict electrode transient voltage waveform (FIG. 3B) when a (single not bi-phasic) step current stimulus is injected with intensity of I₀, and pulse width of t_{catho}. By using a Laplace transform, impedance of the electrode model and the cathodic stimulus is expressed as R_{CT}(1+sR_{CT}C_{dl}) and I₀/s, respectively. The resulting voltage can be derived by taking inverse Laplace transform of the product of the impedance-stimulus:

$${V}_{e} = ( {I}_{0} \times {R}_{S} + {I}_{0} \times {R}_{CT} ( 1 - \frac{- t}{{R}_{CT} \times {C}_{dl}} ) ) u ( t ) \qquad ( 1 )$$

I₀R_{S} in Eq. (1) is the transient voltage increase when the instantaneous current is flowing through R_{S}. This voltage can be measured immediately after the stimulus is fired for the estimation of R_{S}. The second term in Eq. (1) results from the stimulus current which charges C_{dl}. As pulse-width increases, this voltage drop approaches I₀R_{CT} and reaches a plateau. After the stimulus is finished, charge stored in C_{dl} is discharged through the resistive paths and the resulting voltage on the electrode gradually diminishes. It can be inferred from Eq. (1) that a stimulus with sufficiently long pulse-width can drive the subsequent voltage increase of the electrode overpotential to approach I₀R_{CT} and to allow a quick derivation of R_{CT}. However, this might also drive the electrode overpotential over the range of its water window, causing electrode or tissue damage. The term “water window” as utilized in regard to electrodes is the electrochemical window (EW) of a substance (e.g., water) as the voltage range between which the substance is neither oxidized nor reduced. This range is important for the efficiency of an electrode, because out of this range, water is electrolyzed. Returning back to the discussion of R_{CT} it should be noted that once electrode stimulus is out of range, then R_{CT} cannot be determined because C_{dl} cannot be estimated based on Eq. (1).

According to the above observation, a more deliberate stimulus waveform is sought for exciting the electrode in order to yield all the parameters of the Randles cell electrode model with less computation and the prevention of electrode/tissue damage (exceeding the water-window). Herein, a bi-phasic current stimulus with interpulse delay for impedance measurement is disclosed with details provided in the following section.

### 3. The Bio-Impedance Measurement Method

By carefully investigating the transient electrode voltage shown in FIG. 3B and FIG. 3C, it can be found that after the initial electrode voltage increment of I₀R_{S}, there is a short period of time in which the electrode voltage is linearly increasing (ΔV in FIG. 3B). This linear voltage increase is due to the pure capacitive current charging C_{dl} and its value depends on the rate of potential change. Based on charge reservation, the voltage increment during this period can be expressed as:

$$Δ V = \frac{{I}_{0} \times t}{{C}_{dl}} \qquad ( 2 )$$

Once electrode overpotential further increases, Faradic current through R_{CT} starts to conduct a relatively large portion of the injected current from the stimulator and the increment of the electrode overpotential becomes non-linear.

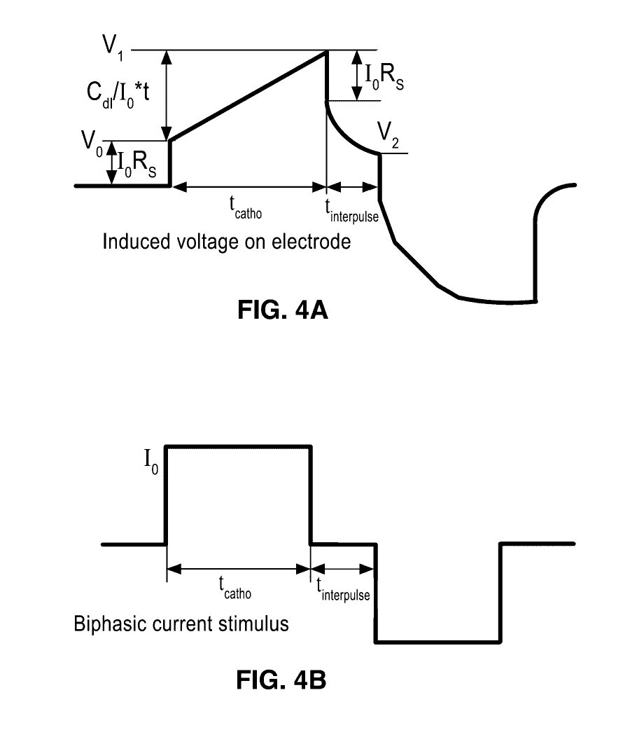

FIG. 4A and FIG. 4B illustrate utilizing a low-intensity, short-period bi-phasic current stimulus with a deliberately inserted interpulse delay in FIG. 4B, with its response seen in FIG. 4A. It is important to note that the pulse width and intensity of the stimulus in FIG. 4A is set to be small so that it does incur pure capacitive charge only which results in a linear increase in electrode overpotential while a conventional current stimulus with higher intensity or long pulse would result in both capacitive and faradic charge transfer as illustrated in FIG. 3B, complicating the process of acquiring the Randles cell electrode model. Using a small and short stimulus can minimize the fraction of Faradic current, allowing the estimation of C_{dl} performed by simply measuring the resulting electrode voltage at the end of the leading pulse (shown as V₁ in FIG. 4A). Subsequently, during interpulse delay tᵢₙₜₑᵣₚᵤₗₛₑ, the charge stored in C_{dl} is passively discharged and the resulting electrode potential Vₑ is given by:

$${V}_{e} = ( {V}_{1} - {I}_{0} {R}_{S} ) ( \frac{- t}{{e}^{{R}_{CT} \times {C}_{dl}}} ) \qquad ( 3 )$$

R_{CT} can thus be derived as:

$${R}_{CT} = \frac{- {t}_{interpulse}}{( {C}_{dl} ln ( \frac{{V}_{e}}{{V}_{1} - {I}_{0} {R}_{S}} ) )} \qquad ( 4 )$$

Insertion of the interpulse delay provides a controlled discharge time and a known timing to sample the electrode potential. Once the electrode voltage is acquired at the end of the interpulse period (shown as V₂ in FIG. 4A), R_{CT} can be determined. Finally, a compensating pulse, seen in the latter half of FIG. 4B is applied to maintain charge balance. Otherwise, accumulated residual charge might result in a DC offset at the electrode and the DC offset might affect the Faradic process, such as affecting R_{CT}, when frequent monitoring of the electrode impedance is performed.

### 4. Test Set-Up

The disclosed bio-impedance measurement technique is targeted at applications, including neural stimulators that deliver electric charge to activate neurons whose operation can be benefited in response to determining bio-impedance at the electrode-electrolyte/tissue interface.

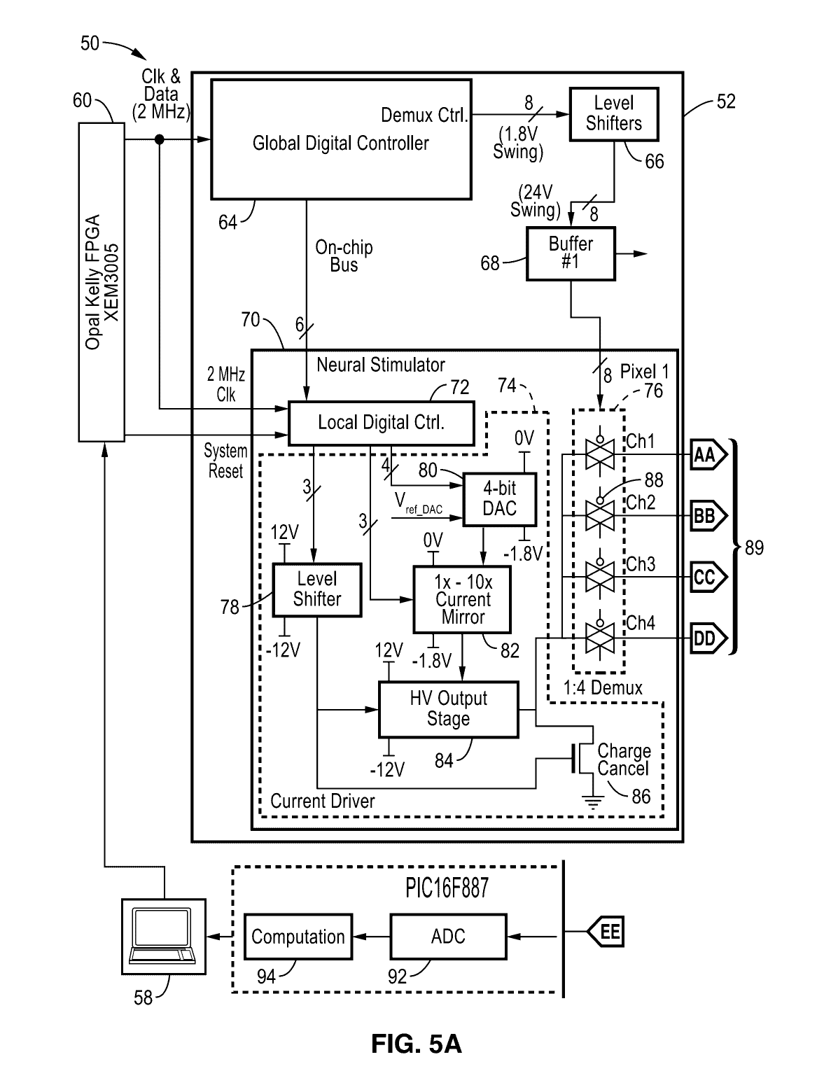

FIG. 5A and FIG. 5B are an example embodiment 50 of a multi-channel neural stimulator utilizing a system-on-chip (SoC) 52 which we developed to generate bi-phasic current stimulus with programmable pulse polarity, intensity, pulse width, and interpulse delay to a group of electrodes 54, such as comprising stimulus electrodes 55a, and a ground electrode 55b. By way of example and not limitation, the electrodes may comprise Ag—AgCl electrodes. Control electronics 56 are shown for registering information from SoC output, which by way of example is also seen coupled to a display device (i.e., oscilloscope).

A FPGA 60 was programmed to send stimulation command to SoC 52. One of ordinary skill in the art will recognize that the FPGA can be replaced by other circuitry, such as processors (MCU, DSP, ASIC, other forms of control circuitry and combinations thereof, without departing from the teachings of the invention. Digital control circuits of the SoC are shown by example with global digital controller 64, level shifters 66, and a first buffer 68 (within multiple buffers as desired) to decode commands and control neural stimulator 70, which is configured to generate a desired current stimulus. Neural stimulator 70 is shown with local digital control 72, a current driver 74, and a demultiplexer 76. The current driver 74 of the stimulator is depicted in this example as comprising a level shifter 78 for translating logic levels for controlling a high voltage (HV) output stage 84, and charge canceling circuit (e.g., transistor) 86. Bits from local control circuit also drive a digital-to-analog (DAC) converter 80 (e.g., 4-bit DAC) whose output drives a current mirror 82, whose output controls the HV output stage 84. Each output HV output stage is connected to 1-to-4 demultiplexer 76, which expands the number of the output channel of the stimulator (i.e., 40 HV output stages build a 160 channel stimulator). Demultiplexer 76 is shown with high voltage drivers/buffers 88 directed to outputs 89, configured for coupling to the electrodes.

Outputs are captured and processed by circuit 56, depicted as comprising a multiplexer 90, analog-to-digital converter (ADC) 92, and a circuit 94 for processing the measured waveform information into a bio-impedance measurement. The processing of digital outputs from the ADC into bio-impedance measurements can be performed by different forms of digital circuitry, such as any desired combination of discrete logic, programmable arrays, application specific integrated circuits, or programming elements. In the example shown, a microcontroller (e.g., PIC16F887 from Microchip Tech. Inc.) is utilized for multiplexing 90 the acquired transient electrode voltage, converting the analog signal to digital 92 (e.g., built-in 10-bit ADC), and for processing the signals to determine bio-impedance. In the example shown, the ADC was set to sample only three voltages (V₀, V₁, and V₂). The sampling operation of the microcontroller in this example is triggered by a synchronization signal from the SoC, in which the synchronization signal was implemented using unused stimulation channel, although these elements can be synchronized using any desired synchronizing circuitry (e.g., clocks, timers, counters, digital logic, other electronic circuits and combinations thereof). Output from circuit 56 is shown for capture and/or display on an external display 58, and/or combination of computer processor and display. An oscilloscope 62 was also used to monitor the evoked potential during stimulation.

It should be appreciated that collecting and processing to arrive at bio-impedance measurements according to the presented technology can be readily implemented within various forms of digital circuitry. It should also be appreciated that such data processing is most readily implemented utilizing one or more computer processor devices (e.g., CPU, microprocessor, microcontroller, computer enabled ASIC, etc.) and associated memory (e.g., RAM, DRAM, NVRAM, FLASH, computer readable media, etc.) whereby instruction codes (programming) stored in the memory and executable on the processor perform the steps of the various process methods described herein. The presented technology is non-limiting with regard to memory and computer-readable media, insofar as these are non-transitory, and thus not constituting a transitory electronic signal.

In order to validate the proposed impedance measurement method, two verification tests were conducted. In the first tests, the proposed method was applied onto an emulated Randles cell made of discrete components with known values. In the second test, the impedance of a custom-made electrode developed at UCLA was evaluated. The stimulation electrodes and an Ag—AgCl reference electrode (e.g., P-BMP-1, ALA scientific instruments, NY) were dipped into a phosphate buffered saline (PBS) solution (concentration of 0.9% sodium chloride). Meanwhile, the impedance of the electrode was also measured using the same set-up through an impedance analyzer (HP 4194A) for verification and comparison.

### 5. Experimental Results and Discussion

The values of each discrete component of the emulated Randles cell R_{CT}, R_{S}, C_{dl}) are 100 kΩ, 10 kΩ, and 30 nF, respectively. Bi-phasic stimuli was applied with an intensity of 10 μA and 100 μA, pulse width of 1 ms, and interpulse delay of 1 ms to this circuit model and the demanded resulting voltages were measured.

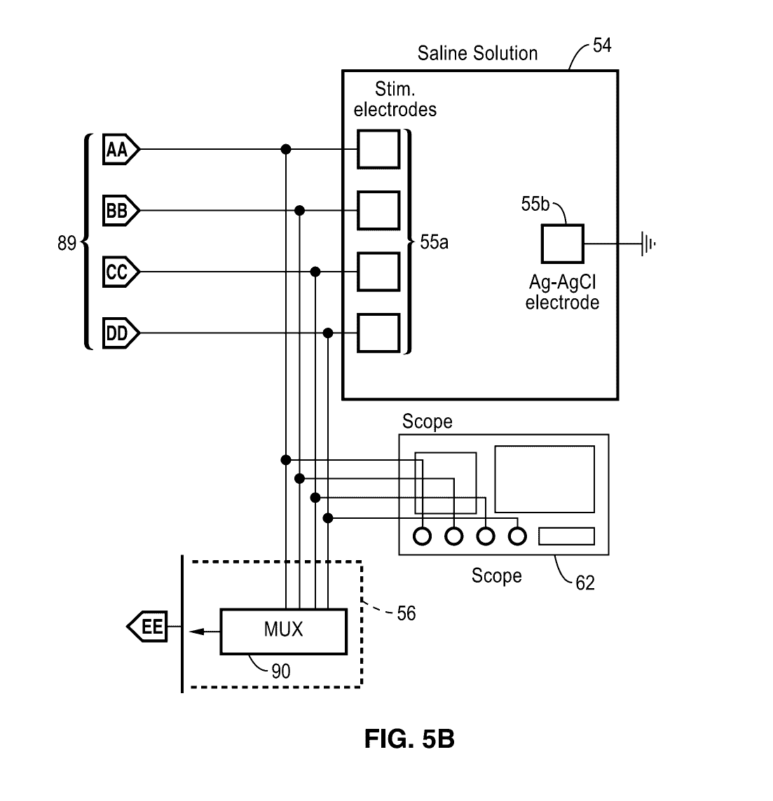

FIG. 6A and FIG. 6B depict measured waveforms of two respective resulting electrode voltages and the estimated component values are shown in these plots as R_{CT}=96.7 kΩ, R_{S}=12 kΩ, C_{dl}=32 nF at 10 μA, and R_{CT}=74.3 kΩ, R_{S}=10.25 kΩ, C_{dl}=41 nF at 100 μA). It can be seen that using small stimulus current provides a more accurate result, while a larger discrepancy from the nominal value of these R_{CT} and C_{dl} is exhibited when utilizing a large stimulus. There is also a slight inconsistency in the estimation of R_{S}. This is possibly due to the non-linearity of the stimulator driver.

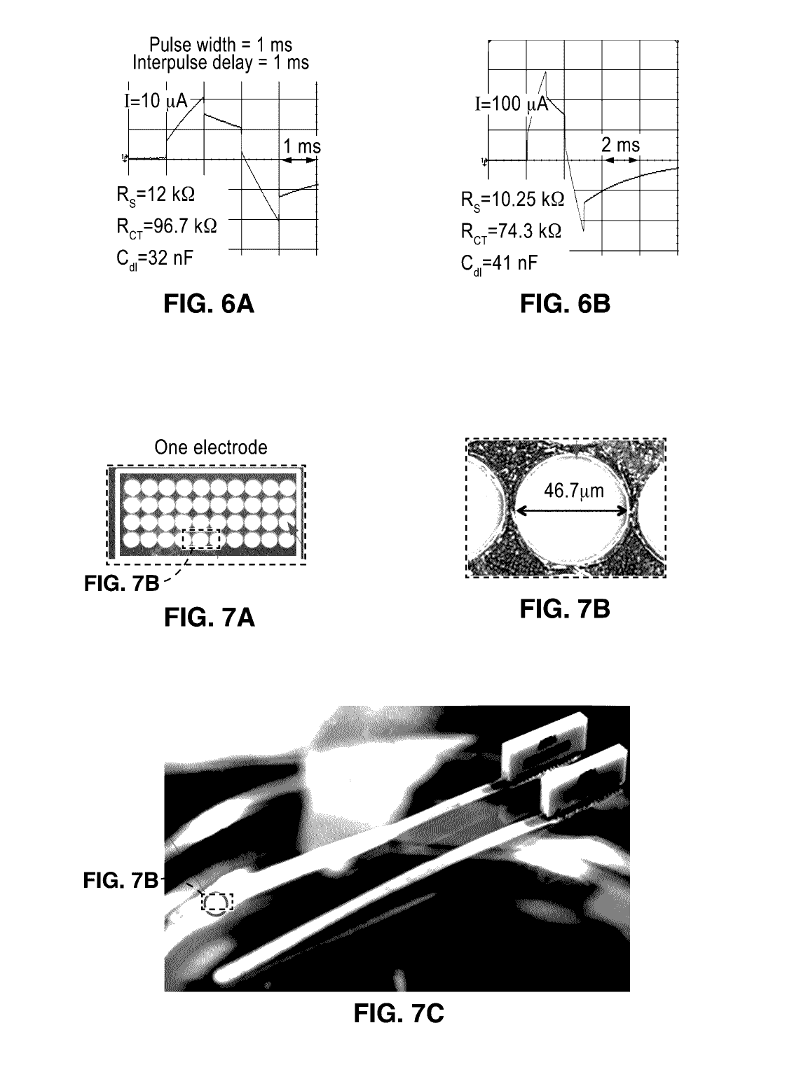

FIG. 7A through FIG. 7C depict a 3×9 platinum polyimide electrode array utilized upon further evaluation of the disclosed technique. FIG. 7A depicts a single electrode of this electrode array, with FIG. 7B depicting one contact of the electrode shown with a diameter of 46.7 μm. In FIG. 7C one can see the entire electrode structure. Impedance was measured of a 3×9 platinum electrode array made on a flexible polyimide substrate. An Omnectics Connector (A79026-001, Omnetics connectors Corp., NM) was used to connect the electrode to the stimulator output. Each single electrode has an area of approximately 200 μm×500 μm with 40 exposed circular regions. R_{CT}, R_{S}, and C_{dl} of the electrode were first characterized and extrapolated as approximately 1.8 kΩ, 15 kΩ, and 176 nF using HP 4194A. Subsequently, bi-phasic stimulus was injected into the electrode.

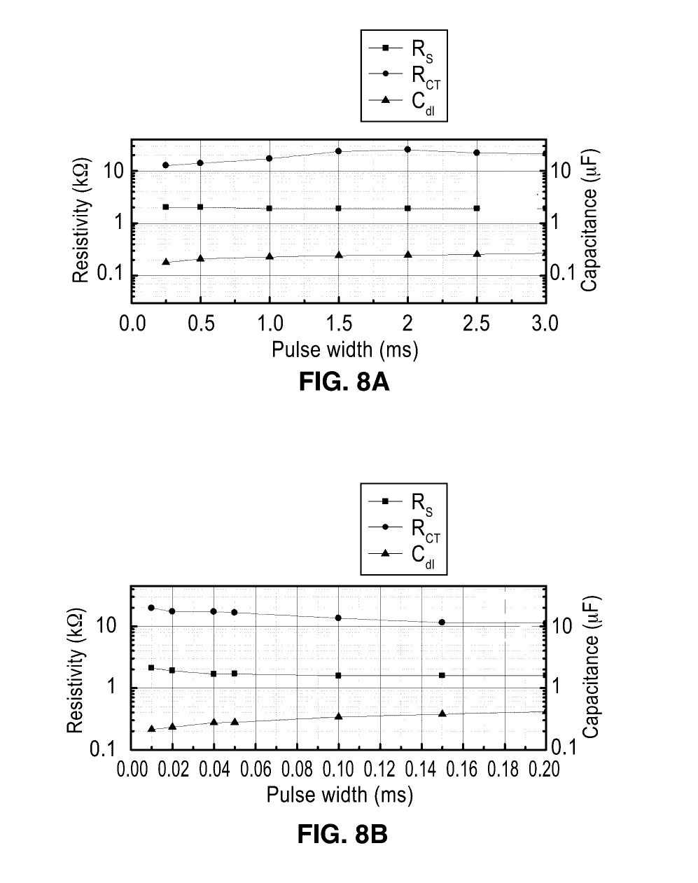

FIG. 8A and FIG. 8B depict estimated circuit parameters of the electrode based on varied stimulus pulse width and stimulus intensity, respectively. It can be seen that estimated R_{S} is in the range of R_{S} 1.9-2.0 kΩ, close to the results from HP4194A. However, as stimulus pulse width and intensity increases, more charge is delivered to the electrode to escalate the electrode overpotential. Therefore, Faradic current gradually increase and it affects the estimation of C_{dl} and R_{CT}. The result and observation imply that using a small stimulus current is preferred in order to accurately estimate parameters of the equivalent circuits model of the electrode. It should also be noted that there is deviation in our measured R_{CT} and C_{dl}, compared with results from HP 4191A. This is possibly due to the fact that large signal analysis is being performed, instead of small signal analysis.

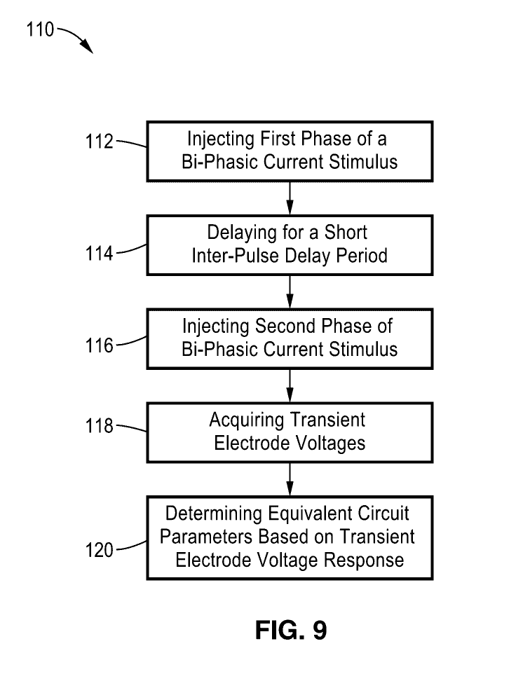

FIG. 9 illustrates an example embodiment 110 of bio-impedance measurement of the present disclosure. A bi-phasic current stimulus is seen being injected having a first phase 112, an inter-pulse delay 114, and a second phase 116. Transient electrode voltages are registered 118, such as at least three selected points along the first phase and inter-pulse delay (e.g., beginning and end of first phase and end of inter-pulse delay). Once voltages are converted to digital signals they are processed 120 to determine equivalent circuit parameters.

The material of the tested electrode in at least one embodiment is platinum that is known to have a pseudo-capacity. However, for a capacitive electrode, such as titanium nitride and tantalum oxide, the proposed method can also be applied to estimate C_{dl} and R_{S}. Moreover, unlike other impedance measurement approaches used in implantable neural stimulator, the proposed method can yield values for both C_{dl} and R_{CT}, instead of R_{S} only. With the knowledge of C_{dl} and R_{CT}, an upper safe bound of the stimulus intensity and pulse width can be set to ensure the electrode over-potential does not exceed its water window.

### 6. Conclusion

The bi-phasic current excitation is disclosed to measure and estimate the equivalent circuits parameters of the Randles cell electrode model. A proof-of-concept system made of a stimulator SoC and a microcontroller/FPGA were implemented to generate the required stimulus and to perform electrode voltage acquisition. Leveraging on the dominating capacitive charging characteristic of the electrode when the electrode overpotential is small, double layer capacitance can be yielded by injecting a small current and measuring the electrode voltage. Through the known double layer capacitance and sampling of electrode voltage, the Faradic charge transfer resistance can be derived through the insertion of a pre-determined discharge time. The electrode transient voltage needs to be sampled only three times and does not require sophisticated computation and hardware, making this approach attractive for implantable stimulators and commercial neural stimulators.

In addition, the measured electrode transient voltage or said bio-impedance can be used as a novel means to monitor/track the smooth muscle activity of gastrointestinal track or vascular blood vessel, providing viable physiological signals.

Embodiments of the present technology may be described with reference to flowchart illustrations of methods and systems according to embodiments of the technology, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that “programming” as used herein refers to one or more instructions that can be executed by a processor to perform a function as described herein. The programming can be embodied in software, in firmware, or in a combination of software and firmware. The programming can be stored local to the device in non-transitory media, or can be stored remotely such as on a server, or all or a portion of the programming can be stored locally and remotely. Programming stored remotely can be downloaded (pushed) to the device by user initiation, or automatically based on one or more factors. It will further be appreciated that as used herein, that the terms processor, central processing unit (CPU), and computer are used synonymously to denote a device capable of executing the programming and communication with input/output interfaces and/or peripheral devices.

From the description herein, it will be appreciated that that the present disclosure encompasses multiple embodiments which include, but are not limited to, the following:

1. A bio-impedance measuring apparatus, comprising: (a) an electrode stimulus circuit configured for generating a low-intensity bi-phasic current stimulus to an attached electrode; (b) wherein said bi-phasic current stimulus comprises a first phase of a first polarity, an interphase delay, and followed by a second phase of a second polarity; (c) an analog to digital converter configured for coupling to said electrode for registering voltage waveforms arising in response to said bi-phasic current stimulus; (d) at least one processor; (e) a memory storing instructions executable by the said least one processor; (f) said instructions when executed by the said at least one processor performing steps comprising: (f)(i) acquiring transient electrode voltages at multiple points during said bi-phasic current stimulus; and (f)(ii) determining parameters of electrode equivalent circuit in response to analyzing said transient electrode voltages with respect to said bi-phasic current stimulus and its inter-pulse delay.

2. The apparatus of any preceding embodiment, wherein said bio-impedance are determined by determining equivalent circuit parameters of an electrode at the electrode-electrolyte/tissue interface.

3. The apparatus of any preceding embodiment, wherein said bio-impedance comprises impedance at the electrode-electrolyte/tissue interface in a biological organism or system.

4. The apparatus of any preceding embodiment, wherein said multiple points to acquire voltages comprises at least three position along said bi-phasic current stimulus.

5. The apparatus of any preceding embodiment, wherein said multiple points for acquiring voltages comprise (i) start of first phase of current application, (ii) end of first phase, (iii) end of interpulse delay.

6. The apparatus of any preceding embodiment, wherein tissue-solution resistance R_{S} is estimated in response to measuring transient voltage increase in response to application of instantaneous current in said bi-phasic current stimulus.

7. The apparatus of any preceding embodiment, wherein double layer capacitance C_{dl} is estimated based on initial pure capacitive charging of the stimulating electrode.

8. The apparatus of any preceding embodiment, wherein said equivalent circuit for the electrode at the electrode-electrolyte/tissue interface is modeled as a Randles cell, having charge transfer resistance R_{CT}, a double layer capacitance C_{dl}, and tissue-solution resistance R_{S}.

9. The apparatus of any preceding embodiment, wherein utilizing a low-intensity stimulus allows estimation of double layer capacitance C_{dl} in an electrode, in response to capacitive charge-injection being dominant when electrode overpotential is small.

10. The apparatus of any preceding embodiment, wherein during said interpulse delay a controlled discharge occurs from which charge transfer resistance R_{CT} is determined.

11. The apparatus of any preceding embodiment, wherein said apparatus is configured for integration into implantable or commercial neural stimulator systems.

12. The apparatus of any preceding embodiment, wherein determination of bio-impedance can be utilized for monitoring propagation of smooth muscle contraction/relaxation waves.

13. The apparatus of any preceding embodiment, wherein said low-intensity bi-phasic current stimulus is time interleaved for use as a biomarker to monitor smooth muscle propagating activity.

14. The apparatus of any preceding embodiment, wherein said apparatus is configured for supporting simultaneous electrical stimulation and recording through the attached electrode.

15. A method for measuring bio-impedance, comprising: (a) injecting a single low-intensity bi-phasic current stimulus to an stimulus electrode configured for use within a biological system; (b) incorporating an inter-pulse delay between the first and second phases of the current stimulus; (c) acquiring transient electrode voltage at multiple temporal locations along the bi-phasic current stimulus; and (d) determining equivalent circuit parameters of an electrode, at the electrode-electrolyte/tissue interface, based on transient electrode voltage across said multiple temporal locations.

16. The method of any preceding embodiment, wherein said bio-impedance are determined by determining equivalent circuit parameters of an electrode at the electrode-electrolyte/tissue interface.

17. The method of any preceding embodiment, wherein said bio-impedance is comprises impedance at the electrode-electrolyte/tissue interface in a biological organism or system.

18. The method of any preceding embodiment, wherein said multiple temporal locations comprise at least positions along said bi-phasic current stimulus.

19. The method of any preceding embodiment, wherein said multiple temporal locations comprise taking voltage measurements at: (i) start of first phase current application, (ii) end of first phase current application, and (iii) end of interpulse delay.

20. The method of any preceding embodiment, wherein tissue-solution resistance R_{S} is estimated in response to measuring transient voltage increase in response to application of instantaneous current in said bi-phasic current stimulus.

21. The method of any preceding embodiment, wherein double layer capacitance C_{dl} is estimated based on initial pure capacitive charging of the stimulating electrode.

22. The method of any preceding embodiment, wherein said equivalent circuit for the electrode at the electrode-electrolyte/tissue interface is modeled as a Randles cell, having charge transfer resistance R_{CT}, a double layer capacitance C_{dl}, and tissue-solution resistance R_{S}.

23. The method of any preceding embodiment, wherein utilizing a low-intensity stimulus allows estimation of double layer capacitance C_{di} in an electrode, since capacitive charge-injection dominates when electrode overpotential is small.

24. The method of any preceding embodiment, wherein during said interpulse delay a controlled discharge occurs from which charge transfer resistance R_{CT} is determined.

25. The method of any preceding embodiment, wherein said method is applicable for integration within implantable or commercial neural stimulator systems.

26. The method of any preceding embodiment, wherein determination of bio-impedance can be utilized for monitoring propagation of smooth muscle contraction/relaxation waves.

27. The method of any preceding embodiment, wherein said low-intensity bi-phasic current stimulus is time interleaved for use as a biomarker to monitor smooth muscle propagating activity.

28. The method of any preceding embodiment, wherein said apparatus is configured for supporting simultaneous electrical stimulation and recording through the attached electrode.

29. A method for measuring bio-impedance, comprising determining the equivalent circuit of an electrode by injecting a single low-intensity bi-phasic current stimulus with inter-pulse delay and acquiring the transient electrode voltage at three well-specified timing.

30. An apparatus for measuring bio-impedance, comprising: an electrode; a computer processor; and a memory storing a computer program executable by the computer processor; said computer program configured to, when executed, determine the equivalent circuit of the electrode by injecting a single low-intensity bi-phasic current stimulus with inter-pulse delay and acquiring the transient voltage of the electrode at three well-specified times.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A bio-impedance measuring apparatus, comprising:
(a) an electrode stimulus circuit configured for generating a low-intensity bi-phasic current stimulus to an attached electrode;
(b) wherein said bi-phasic current stimulus comprises a first phase of a first polarity, an interphase delay, and is followed by a second phase of a second polarity;
(c) an analog to digital converter configured for coupling to said electrode for registering voltage waveforms arising in response to said bi-phasic current stimulus;
(d) at least one processor; and
(e) a memory storing instructions executable by the at least one processor;
(f) said instructions when executed by the at least one processor performing steps comprising:
(i) acquiring transient electrode voltages at multiple points during said bi-phasic current stimulus; and
(ii) determining parameters of electrode equivalent circuit in response to analyzing said transient electrode voltages with respect to said bi-phasic current stimulus and its inter-pulse delay.

2. The apparatus as recited in claim 1, wherein said bio-impedance are determined by determining equivalent circuit parameters of an electrode at the electrode-electrolyte/tissue interface.

3. The apparatus as recited in claim 1, wherein said bio-impedance comprises impedance at the electrode-electrolyte/tissue interface in a biological organism or system.

4. The apparatus as recited in claim 1, wherein said multiple points to acquire voltages comprise at least three positions along said bi-phasic current stimulus.

5. The apparatus as recited in claim 4, wherein said multiple points for acquiring voltages comprise (i) start of first phase of current application, (ii) end of first phase, (iii) end of interpulse delay.

6. The apparatus as recited in claim 1, wherein tissue-solution resistance R_{S} is estimated in response to measuring transient voltage increase in response to application of instantaneous current in said bi-phasic current stimulus.

7. The apparatus as recited in claim 1, wherein double layer capacitance C_{dl} is estimated based on initial pure capacitive charging of the stimulating electrode.

8. The apparatus as recited in claim 1, wherein said equivalent circuit for the electrode at the electrode-electrolyte/tissue interface is modeled as a Randles cell, having charge transfer resistance R_{CT}, a double layer capacitance C_{dl}, and tissue-solution resistance R_{S}.

9. The apparatus as recited in claim 8, wherein utilizing a low-intensity stimulus allows estimation of double layer capacitance C_{dl} in an electrode, in response to capacitive charge-injection being dominant when electrode overpotential is small.

10. The apparatus as recited in claim 8, wherein during said interpulse delay a controlled discharge occurs from which charge transfer resistance R_{CT} is determined.

11. The apparatus as recited in claim 1, wherein said apparatus is configured for integration into implantable or commercial neural stimulator systems.

12. The apparatus as recited in claim 1, wherein determination of bio-impedance can be utilized for monitoring propagation of smooth muscle contraction/relaxation waves.

13. The apparatus as recited in claim 1, wherein said low-intensity bi-phasic current stimulus is time interleaved for use as a biomarker to monitor smooth muscle propagating activity.

14. The apparatus as recited in claim 1, wherein said apparatus is configured for supporting simultaneous electrical stimulation and recording through the attached electrode.

15. A method for measuring bio-impedance, comprising:
(a) injecting a single low-intensity bi-phasic current stimulus to a stimulus electrode configured for use within a biological system;
(b) incorporating an inter-pulse delay between the first and second phases of the current stimulus;
(c) acquiring transient electrode voltage at multiple temporal locations along the bi-phasic current stimulus; and
(d) determining equivalent circuit parameters of an electrode, at the electrode-electrolyte/tissue interface, based on transient electrode voltage across said multiple temporal locations.

16. The method as recited in claim 15, wherein said bio-impedance is determined by determining equivalent circuit parameters of an electrode at the electrode-electrolyte/tissue interface.

17. The method as recited in claim 15, wherein said bio-impedance comprises impedance at the electrode-electrolyte/tissue interface in a biological organism or system.

18. The method as recited in claim 15, wherein said multiple temporal locations comprises at least three positions along said bi-phasic current stimulus.

19. The method as recited in claim 18, wherein said multiple temporal locations comprise taking voltage measurements at: (i) start of first phase current application, (ii) end of first phase current application, and (iii) end of interpulse delay.

20. The method as recited in claim 15, wherein tissue-solution resistance R_{S} is estimated in response to measuring transient voltage increase in response to application of instantaneous current in said bi-phasic current stimulus.

21. The method as recited in claim 15, wherein double layer capacitance C_{dl} is estimated based on initial pure capacitive charging of the stimulating electrode.

22. The method as recited in claim 15, wherein said equivalent circuit for the electrode at the electrode-electrolyte/tissue interface is modeled as a Randles cell, having charge transfer resistance R_{CT}, a double layer capacitance C_{dl}, and tissue-solution resistance R_{S}.

23. The method as recited in claim 22, wherein utilizing a low-intensity stimulus allows estimation of double layer capacitance C_{dl} in an electrode, since capacitive charge-injection dominates when electrode overpotential is small.

24. The method as recited in claim 22, wherein during said interpulse delay a controlled discharge occurs from which charge transfer resistance R_{CT} is determined.

25. The method as recited in claim 15, wherein said method is applicable for integration within implantable or commercial neural stimulator systems.

26. The method as recited in claim 15, wherein determination of bio-impedance can be utilized for monitoring propagation of smooth muscle contraction/relaxation waves.

27. The method as recited in claim 15, wherein said low-intensity bi-phasic current stimulus is time interleaved for use as a biomarker to monitor smooth muscle propagating activity.

28. The method as recited in claim 15, wherein said method is configured for supporting simultaneous electrical stimulation and recording through the attached electrode.

29. A method for measuring bio-impedance, comprising determining the equivalent circuit of an electrode by injecting a single low-intensity bi-phasic current stimulus with inter-pulse delay and acquiring the transient electrode voltage at three well-specified timing.

30. An apparatus for measuring bio-impedance, comprising:
an electrode;
a computer processor; and
a memory storing a computer program executable by the computer processor;
said computer program configured to, when executed, determine the equivalent circuit of the electrode by injecting a single low-intensity bi-phasic current stimulus with inter-pulse delay and acquiring transient voltage of the electrode at three well-specified times.
